(19) United States
(12) Patent Application Publication
(10) Pub. No.: US 2019/0359603 A1
(43) Pub. Date: Nov. 28, 2019

(54) METHOD FOR PREPARING THIOLACTONES, THIOLACTONES OBTAINED BY SAID METHOD AND USES THEREOF

(71) Applicants: CENTRE NATIONAL DE LA RECHERCHE SCIENTIFIQUE, PARIS (FR); UNIVERSITE TOULOUSE III - Paul Sabatier, Toulouse (FR)

(72) Inventors: Olivier COUTELIER, AUZEVILLE TOLOSANE (FR); Mathias DESTARAC, BALMA (FR); Marvin LANGLAIS, TOULOUSE (FR)

(21) Appl. No.: 16/477,424

(22) PCT Filed: Jan. 16, 2018
(86) PCT No.: PCT/FR2018/050099

§ 371 Date: Jul. 11, 2019

(30) Foreign Application Priority Data
Jan. 18, 2017 (FR) 1750384

(51) Int. Cl. C07D 409/06; C07D 333/32

(52) U.S. Cl. CPC C07D 409/06; C07D 333/32

(57) ABSTRACT

The present invention relates to a process for the preparation of substituted thiolactones of formula (I) or of substituted thiolactones of formula (I′) which are capable of being obtained by the implementation of this process, and to the use of substituted thiolactones of formula (I) or of formula (I′) in the preparation of polymers or in the functionalization of particles, of flat surfaces or of polymers.

The present invention relates to the field of thiolactones.

More particularly, the present invention relates to a process for the preparation of substituted thiolactones of formula (I) or of substituted thiolactones of formula (I′) which are capable of being obtained by the implementation of this process, and to the use of substituted thiolactones of formula (I) or of formula (I′) in the preparation of polymers or in the functionalization of particles, of flat surfaces or of polymers.

Thiolactones are heterocyclic compounds which are analogues of lactones, in which an oxygen atom is replaced with a sulfur atom. The sulfur atom is located in the ring and is adjacent to a carbonyl group. The heterocycle of the thiolactones can be substituted by at least one chemical group, in particular by an alkyl or aryl group.

Several processes for the synthesis of thiolactones have already been provided.

Korte et al. [Chem. Ber., 1961, 94, 1966-1976] have, for example, provided either for carrying out a thermal cyclization of a mercaptocarboxylic acid carrying an alkyl substituent or for directly substituting a thiolactone with an alkyl radical in the presence of an alkyl halide (R—X) group and of a lithium dialkylamide (LiNR′₂). These two synthesis routes can be represented by the following reaction scheme (1):

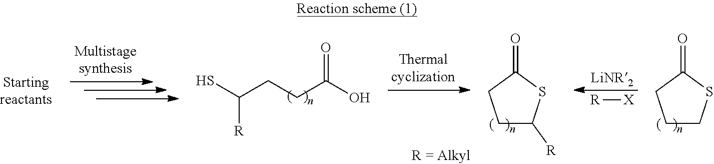

According to the thermal cyclization synthesis route, only the final cyclization stage is general, the preceding stages resulting in the mercaptocarboxylic acid and the reactants employed being specific to the type of R group which it is desired to introduce onto the heterocycle. Moreover, these two synthesis routes do not make it possible to introduce substituents other than alkyl groups.

A more recent synthesis process makes it possible to access thiolactones possessing alkyl or aryl groups (Filippi et al., Tet. Lett., 2006, 47, 6067-6070). This process is based on a method for the catalytic isomerization of a thionolactone to give a thiolactone in the presence of boron trifluoride (BF₃) and of diethyl ether (Et₂O) in an organic solvent, such as toluene, at reflux, according to the following reaction scheme (2):

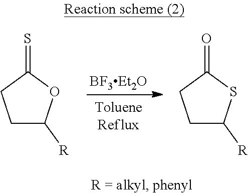

However, this process uses a catalyst of Lewis acid type (e.g., boron trifluoride) and cannot be readily used for the synthesis of thiolactones carrying substituents other than alkyl or phenyl groups, such as organic functional groups which are complex and/or incompatible with this type of catalyst. Moreover, the isolated yields of the thiolactones are often low. Finally, this process requires the synthesis of the starting thionolactones from the corresponding lactones.

There thus exists a need for a process which makes it possible to synthesize thiolactones substituted by varied functional groups in a flexible and simple manner, and according to a process which is both efficient and economical.

A first subject-matter of the present invention is thus a process for the preparation of substituted thiolactones of following formula (I):

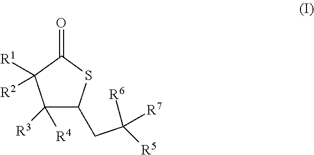

in which:
R¹, R², R³ and R⁴, which are identical or different, represent a hydrogen atom or a group chosen from alkyl, acyl, aryl, heteroaryl, saturated or unsaturated cycloalkyl and saturated or unsaturated heterocycloalkyl groups, it also being possible for the R¹, R², R³ and R⁴ radicals to together form a saturated or unsaturated cycloalkyl or heterocycloalkyl group or an aryl or heteroaryl group; and
R⁵, R⁶ and R⁷, which are identical or different, are:

(a) chosen from a hydrogen atom, a cyano (CN) group, an alkyl group, an acyl group, an aryl group, a heteroaryl group, an aralkyl group, a saturated or unsaturated cycloalkyl group, a saturated or unsaturated heterocycloalkyl group and a phthalimido group, it being possible for said alkyl, acyl, aryl, heteroaryl, aralkyl, saturated or unsaturated cycloalkyl, saturated or unsaturated heterocycloalkyl and phthalimido groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N⁺R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from CI, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or

(b) such that they together form a polymer chain P¹; or

(c) such that R⁵ is different from the other two groups R⁶ and R⁷, R⁶ and R⁷ have the same definitions as in the alternative (a), and R⁵ is a thiolactone radical of following formula:

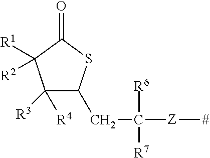

in which R¹, R², R³ and R⁴ have the same meanings as in the formula (I) above, R⁶ and R⁷ have the same definitions as in the alternative (a), Z represents a divalent group chosen from a carbonyl group, a carbonate group, an alkylene group and an arylene group, and the sign # represents the point of attachment of the thiolactone radical to the —CR⁶R⁷CH₂-thiolactone radical of the compound of formula (I);

said process being characterized in that it comprises at least the following stages:

1) a stage during which a xanthate of following formula (II):

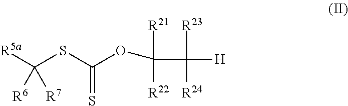

in which:
R²¹, R²², R²³ and R²⁴, which are identical or different, represent a hydrogen atom or a group chosen from alkyl, acyl, aryl, heteroaryl, alkenyl, alkynyl, saturated or unsaturated cycloalkyl and saturated or unsaturated heterocycloalkyl groups, it also being possible for the R²¹, R²², R²³ and R²⁴ radicals to together form a saturated or unsaturated cycloalkyl or heterocycloalkyl group or an aryl or heteroaryl group;

and
R^{5a}, R⁶ and R⁷, which are identical or different, are:

(a′) chosen from a hydrogen atom, a cyano (CN) group, an alkyl group, an acyl group, an aryl group, a heteroaryl group, an aralkyl group, a saturated or unsaturated cycloalkyl group, a saturated or unsaturated heterocycloalkyl group and a phthalimido group, it being possible for said alkyl, acyl, aryl, heteroaryl, aralkyl, saturated or unsaturated cycloalkyl, saturated or unsaturated heterocycloalkyl and phthalimido groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N+R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from CI, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or

(b′) such that they together form a polymer chain P¹; or

(c′) such that R^{5a} is different from the other two groups R⁶ and R⁷, R⁶ and R⁷ have the same definitions as in the alternative (a′) and R^{5a} is a xanthate radical of formula:

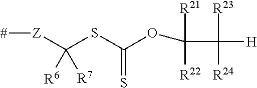

in which R²¹, R²², R²³ and R²⁴ have the same meanings as in the formula (II) above, R⁶ and R⁷ have the same definitions as in the alternative (a′), Z represents a divalent group chosen from a carbonyl group, a carbonate group, an alkylene group and an arylene group, and the sign # represents the point of attachment of the xanthate radical to the —CR⁶R⁷-xanthate radical of the compound of formula (II);

is reacted, in the presence of a radical initiator, with a monomer comprising at least one ethylenic unsaturation of following formula (III):

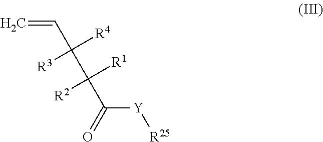

in which:
R²⁵ represents a group chosen from alkyl, acyl, aryl, heteroaryl, aralkyl, saturated or unsaturated cycloalkyl and saturated or unsaturated heterocycloalkyl groups;
Y is an oxygen atom or an NR²⁶ radical in which R²⁶ represents a hydrogen atom or an alkyl group, and preferably an oxygen atom; and
R¹, R², R³ and R⁴ have the same meanings as in the formula (I) above;

to form a monoadduct of following formula (IV):

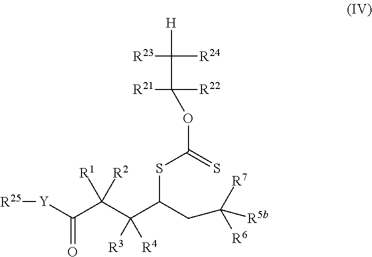

in which:
R¹, R², R³ and R⁴ have the same meanings as in the formula (I) above;
R²¹, R²², R²³ and R²⁴ have the same meanings as in the formula (II) above; and
R²⁵ has the same meaning as in the formula (III) above; and
R^{5b}, R⁶ and R⁷, which are identical or different, are:

(a″) chosen from a hydrogen atom, a cyano (CN) group, an alkyl group, an acyl group, an aryl group, a heteroaryl group, an aralkyl group, a saturated or unsaturated cycloalkyl group, a saturated or unsaturated heterocycloalkyl group and a phthalimido group, it being possible for said alkyl, acyl, aryl, heteroaryl, aralkyl, saturated or unsaturated cycloalkyl, saturated or unsaturated heterocycloalkyl and phthalimido groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N+R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from CI, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or

(b″) such that they together form a polymer chain P¹; or

(c″) such that R^{5b} is different from the other two groups R⁶ and R⁷, R⁶ and R⁷ have the same definitions as in the alternative (a″) and R^{5b} is a monoadduct radical of following formula:

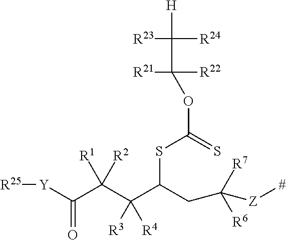

in which R¹, R², R³, R⁴, R²¹, R²², R²³, R²⁴, Y and R²⁵ have the same meanings as in the formula (IV) above, R⁶ and R⁷ have the same definitions as in the alternative (a″), Z represents a divalent group chosen from a carbonyl group, a carbonate group, an alkylene group and an arylene group, and the sign # represents the point of attachment of the monoadduct radical to the —CR⁶R⁷-monoadduct radical of the compound of formula (IV); then

2) a stage of thermolysis of the monoadduct of formula (IV) obtained above in the preceding stage in order to form a corresponding substituted thiolactone of formula (I).

The process for the preparation of the substituted thiolactones of formula (I) in accordance with the invention can be represented by the following reaction scheme (3):

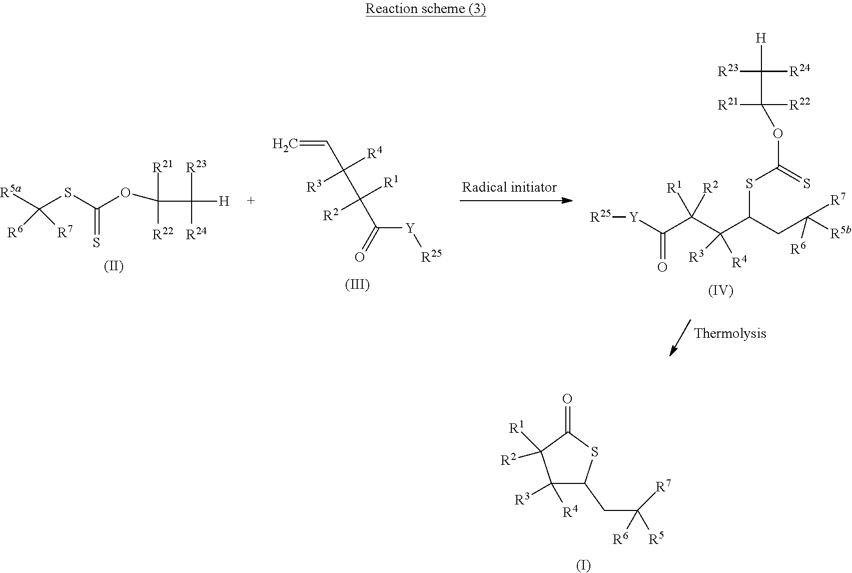

By virtue of the process in accordance with the present invention and as described above, it is henceforth possible, in a simple and rapid way and with a good yield, to access thiolactones substituted by varied organic groups.

In particular, the process of the invention makes it possible, during the thermolysis stage 2), to access a substituted thiolactone in a simple way in a single stage. The thermolysis 2) employs less aggressive conditions than the conditions generally employed in the prior art, such as the acidic/basic conditions which employ acidic reactants and/or basic reactants in several stages. These acidic/basic conditions are not desired as they do not make it possible to obtain substituted thiolactones having nonresilient chemical groups, such as cyano groups; furthermore, they very often result in poor yields due to the formation of byproducts.

In the process of the invention, the simple heating promotes the cyclization and thus the achievement of a substituted thiolactone with acceptable yields. The alkyl radicals mentioned for R¹, R², R³, R⁴, R⁵, R^{5a}, R^{5b}, R⁶, R⁷, R⁸, R^{8′}, R⁹, R¹⁰, R¹¹, R¹², R¹³, R¹⁴, R¹⁵, R^{15′}, R^{15″}, R¹⁶, R^{16′}, R^{17′}, R¹⁷, R¹⁸, R¹⁹, R^{19′}, R²⁰, R²¹, R²², R²³, R²⁴ and R²⁵ can be linear or branched and substituted or unsubstituted and can comprise from 1 to 12 carbon atoms and preferably from 1 to 6 carbon atoms. They are preferably chosen from methyl, ethyl, n-propyl, isopropyl, n-butyl, 2-butyl, isobutyl, tert-butyl, n-pentyl, isopentyl, neopentyl, tert-pentyl, hexyl, n-octyl, isooctyl, 2-ethyl-1-hexyl, 2,2,4-trimethylpentyl, nonyl, decyl, dodecyl and benzyl radicals. Preference is very particularly given, among such radicals, to any one of the methyl, ethyl, n-propyl, isopropyl or n-butyl radicals.

The alkyl radicals mentioned for R¹, R², R³, R⁴, R⁵, R^{5a}, R^{5b}, R⁶, R⁷, R⁸, R^{8′}, R⁹, R¹⁰, R¹¹, R¹², R¹³, R¹⁴, R¹⁵, R^{15′}, R^{15″}, R¹⁶, R^{16′}, R^{17′}, R¹⁷, R¹⁸, R¹⁹, R^{19′}, R²⁰, R²¹, R²², R²³, R²⁴ and R²⁵ can be fluorinated or perfluorinated.

Within the meaning of the present invention, an acyl group denotes a group of formula —C(═O)-D, in which D denotes a hydrogen atom or a saturated or unsaturated and linear or branched hydrocarbon chain which can comprise from 1 to 12 carbon atoms and preferably from 1 to 6 carbon atoms. Mention may in particular be made, among such acyl groups mentioned for R¹, R², R³, R⁴, R⁵, R^{5a}, R^{5b}, R⁶, R⁷, R²¹, R²², R²³, R²⁴ and R²⁵, of the formyl, acetyl, propanoyl or pivaloyl groups.

Within the meaning of the present invention, aryl group is understood to mean a monocyclic or polycyclic aromatic hydrocarbon group which is optionally monosubstituted or polysubstituted, comprising from 3 to 10 carbon atoms and preferably from 3 to 6 carbon atoms. Mention may in particular made, as aryl radical mentioned for R¹, R², R³, R⁴, R⁵, R^{5a}, R^{5b}, R⁶, R⁷, R¹², R¹³, R¹⁴, R¹⁵, R^{15′}, R^{15″}, R¹⁶, R^{16′}, R¹⁷, R^{17′}, R¹⁸, R¹⁹, R^{19′}, R²⁰, R²¹, R²², R²³, R²⁴ and R²⁵, of the naphthyl, anthranyl, phenanthryl, o-tolyl, p-tolyl, xylyl, ethylphenyl, mesityl and phenyl groups. The phenyl group is particularly preferred among such groups.

Within the meaning of the present invention, the cycloalkyl group is a cyclic group comprising from 3 to 10 carbon atoms and preferably from 3 to 7 carbon atoms. The cycloalkyl group is preferably saturated. Mention may in particular be made, among such cycloalkyl groups mentioned for R¹, R², R³, R⁴, R⁵, R^{5a}, R^{5b}, R⁶, R⁷, R²¹, R²², R²³, R²⁴ and R²⁵, of the cyclopropyl, cyclobutyl, cyclopentyl, cyclohexyl or cycloheptyl groups.

The cycloalkyl groups mentioned for R¹, R², R³, R⁴, R⁵, R^{5a}, R^{5b}, R⁶, R⁷, R²¹, R²², R²³, R²⁴ and R²⁵ can be fluorinated or perfluorinated.

Still within the meaning of the present invention, a heterocycloalkyl group is a cyclic group comprising from 3 to 10 carbon atoms, and preferably from 3 to 6 carbon atoms, and at least one heteroatom chosen from N, O, P, Si and S. The heterocycloalkyl group is preferably saturated. Mention may in particular be made, among such heterocycloalkyl groups mentioned for R¹, R², R³, R⁴, R⁵, R^{5a}, R^{5b}, R⁶, R⁷, R²¹, R²², R²³, R²⁴ and R²⁵, of the oxacyclopropanyl, azacyclopropanyl, thiacyclopropanyl, tetrahydrofuranyl, pyrrolidinyl, tetrahydrothiophenyl, tetrahydropyranyl, piperidinyl, piperazinyl or thiacyclohexyl groups.

The heterocycloalkyl groups mentioned for R¹, R², R³, R⁴, R⁵, R^{5a}, R^{5b}, R⁶, R⁷, R²¹, R²², R²³, R²⁴ and R²⁵ can be fluorinated or perfluorinated.

A heteroaryl group, within the meaning of the present invention, is a monocyclic or polycyclic aromatic group, optionally monosubstituted or polysubstituted, comprising from 3 to 10 carbon atoms, and preferably from 5 to 6 carbon atoms, and at least one heteroatom chosen from N, O, P, Si and S. Mention may in particular be made, among such heteroaryl groups mentioned for R¹, R², R³, R⁴, R⁵, R^{5a}, R^{5b}, R⁶, R⁷, R²¹, R²², R²³, R²⁴ and R²⁵, of the furanyl, thiophenyl, pyrrolyl, pyridinyl, pyranyl, oxazinyl, thiazinyl, pyrimidinyl, piperazinyl or thiinyl groups.

An aralkyl group, within the meaning of the present invention, is a group comprising at least one alkyl radical and at least one aryl radical, said alkyl and aryl radicals being connected via a carbon-carbon bond and said alkyl and aryl radicals having the same definition as that given for the alkyl and aryl radicals above. Mention may in particular be made, as aralkyl group, of the benzyl group.

An alkylene group, within the meaning of the present invention, can be linear or branched and substituted or unsubstituted and can comprise from 1 to 12 carbon atoms and preferably from 1 to 6 carbon atoms.

An arylene group, within the meaning of the present invention, can be monosubstituted or polysubstituted and can comprise from 10 to 30 carbon atoms and preferably from 10 to 20 carbon atoms.

According to the alternative (b) for the formula (I), the R⁵, R⁶ and R⁷ radicals are chosen so that the —CR⁵R⁶R⁷ group forms a polymer chain P¹; according to the alternative (b′) for the formula (II), the R^{5a}, R⁶ and R⁷ radicals are chosen so that the —CR^{5a}R⁶R⁷ group forms a polymer chain P¹; and, according to the alternative (b″) for the formula (IV), the R^{5b}, R⁶ and R⁷ radicals are chosen so that the —CR^{5b}R⁶R⁷ group forms a polymer chain P¹.

According to the present invention, polymer chain P¹ is understood to mean, for the —CR⁵R⁶R⁷, —CR^{5a}R⁶R⁷ and —CR^{5b}R⁶R⁷ groups, any sequence of monomer units obtained by a radical polymerization process controlled by reversible addition-fragmentation (i.e., process also denoted RAFT/MADIX), such as the RAFT/MADIX process described, for example, by Moad et al. [Aust. J. Chem., 2012, 65(8), 985-1076] or by Destarac et al. [ACS Symposium Series, vol. 854, American Chemical Society, 2003. Matyjaszewski, K., Ed. Advances in Controlled/Living Radical Polymerization, page 536], or by atom transfer, such as the ATRP (Atom Transfer Radical Polymerization) process described, for example, by Matyjaszewski et al. [Chem. Rev., 2001, 101(9), 2921-2990], carried out so that the terminal monomer unit connected respectively to the sulfur atom of the thiocarbonylthio group (RAFT/MADIX) or the halogen atom (Cl, Br) for ATRP, is of acrylate type, for example methyl acrylate, or acrylamido type, such as N-isopropylacrylamide.

The polymer chain P¹ can also result from the transformation of a polymer exhibiting at least one terminal —OH or at least one terminal —NH₂ into an appropriate xanthate end.

The polymer chain P¹ can be chosen from a polydimethylsiloxane, a random or block copolymer based on dimethylsiloxane units, a polyethylene oxide, a polypropylene oxide, a random or block copolymer based on ethylene oxide and on propylene oxide, a poly(butylene oxide), a random or block copolymer based on ethylene oxide and on butylene oxide, a polytetramethylene oxide (poly(tetrahydrofuran)), a polylactide, a polycaprolactone, a polyester, a polyethylene, a poly(ethylene-co-butylene) (or hydrogenated polybutadiene), a polypropylene, an oligopeptide, a polypeptide, a polyamide, a polyurethane, a polystyrene and a polymer synthesized by controlled radical polymerization of unsaturated monomers according to techniques known in the state of the art, such as ATRP, NMP (Nitroxide Mediated Polymerization), for example described by Hawker et al. [Chem. Rev., 2001, 101, 3661-3688], RAFT/MADIX, OHMRP (OrganoHeteroatom Mediated living Radical Polymerization), for example described by Yamago et al. [Chem. Rev., 2009, 109, 5051-5068], and the like.

Preferably, at least one of the R²¹ or R²² groups is other than a hydrogen atom.

In a specific embodiment, R²¹, R²², R²³ and R²⁴ represent a hydrogen atom or an alkyl group.

R²¹ (respectively R²²) can be an alkyl group, in particular a methyl group, and R²² (respectively R²¹) can be a hydrogen atom.

Preferably, at least one of the R²³ and R²⁴ groups is other than a hydrogen atom.

More preferably, R²³ (respectively R²⁴) is an alkyl group, in particular a methyl group, and R²⁴ (respectively R²³) is a hydrogen atom or an alkyl group, in particular a methyl group.

Preferably, the R⁵ group is other than a hydrogen atom.

According to a particularly preferred embodiment of the invention, R⁵ is a cyano group or a phthalimido group.

In a preferred embodiment, at least one of the R⁶ or R⁷ groups is a hydrogen atom and advantageously the two R⁶ and R⁷ groups are hydrogen atoms.

In a specific embodiment, R²⁵ is an alkyl group, in particular a methyl group.

In a specific embodiment, R¹, R², R³ and R⁴ represent a hydrogen atom or an alkyl group.

Preferably, R¹ (respectively R²) is an alkyl group, in particular a methyl group, and R² (respectively R¹) is a hydrogen atom.

Preferably, at least one of the R³ or R⁴ groups is a hydrogen atom and more preferably the two R³ and R⁴ groups are hydrogen atoms.

The R²⁶ group is preferably a methyl group.

In the formula (I) as defined above, the alternatives (a) and (b) are preferred and the alternative (a) is even more preferred.

In the formula (II) as defined above, the alternatives (a′) and (b′) are preferred and the alternative (a′) is even more preferred.

In the formula (IV) as defined above, the alternatives (a″) and (b″) are preferred and the alternative (a″) is even more preferred.

According to a particularly advantageous embodiment, the process of the invention results in the formation of a thiolactone of formula (I) chosen from:
3-(4-methyl-5-oxotetrahydrothiophen-2-yl)propanenitrile (TL1), and
2-(2-(4-methyl-5-oxotetrahydrothiophen-2-yl)ethyl)isoindoline-1,3-dione (TL2).

When they are not commercially available, the xanthates of formula (II):

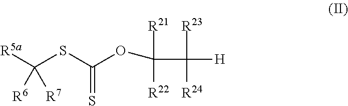

can be obtained according to a process analogous to that used in International Application WO 2004/024681. In particular, they can be obtained according to a process comprising the following stages:

a) reacting, in an organic solvent, an alcohol of following formula (VI):

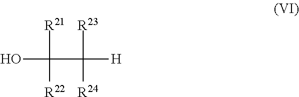

in which the R²¹, R²², R²³ and R²⁴ radicals have the same meanings as in the xanthates of formula (II) above,

with carbon disulfide (CS₂) in the presence of a base, in order to obtain a salt of following formula (VII):

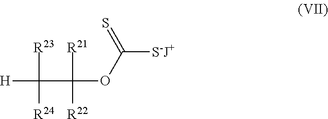

in which the R²¹, R²², R²³ and R²⁴ radicals have the same meanings as in the xanthates of formula (II) above and J⁺ is a cation chosen from the cations of alkali metals, such as a K⁺ or Na⁺ cation, then

b) reacting the compound of formula (VII) obtained in stage a) above with a compound of following formula (VIII):

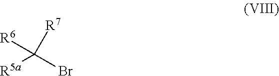

in which R^{5a}, R⁶ and R⁷ have the same meanings as in the xanthate of formula (II) above, in order to obtain a corresponding xanthate of formula (II).

The first stage a) of preparation of a salt of formula (VII) is preferably carried out at ambient temperature, especially in an organic solvent, such as tetrahydrofuran, and by using in particular a strong base, preferably potassium hydroxide. The duration of stage a) is generally from 20 to 24 hours approximately.

The second stage b) of preparation of a xanthate of formula (II) is preferably carried out in an organic solvent, such as acetone, and especially in an ice bath, the addition reaction of the compound of formula (VIII) being highly exothermic. Once the addition of the compound of formula (VIII) is complete, the reaction is preferably carried out at ambient temperature, especially for a duration of 2 to 4 hours approximately. When the reaction is complete, the xanthate of formula (II) thus obtained can be filtered, and then the filtrate is preferably concentrated under vacuum. The xanthate of formula (II) can subsequently be used in the process in accordance with the present invention without additional purification.

When the —CR^{5a}R⁶R⁷ group is a polymer chain P¹, the xanthate of formula (II) can be obtained by RAFT/MADIX polymerization of monomers or by organic synthesis of a polymer-xanthate according to the following reaction scheme (4):

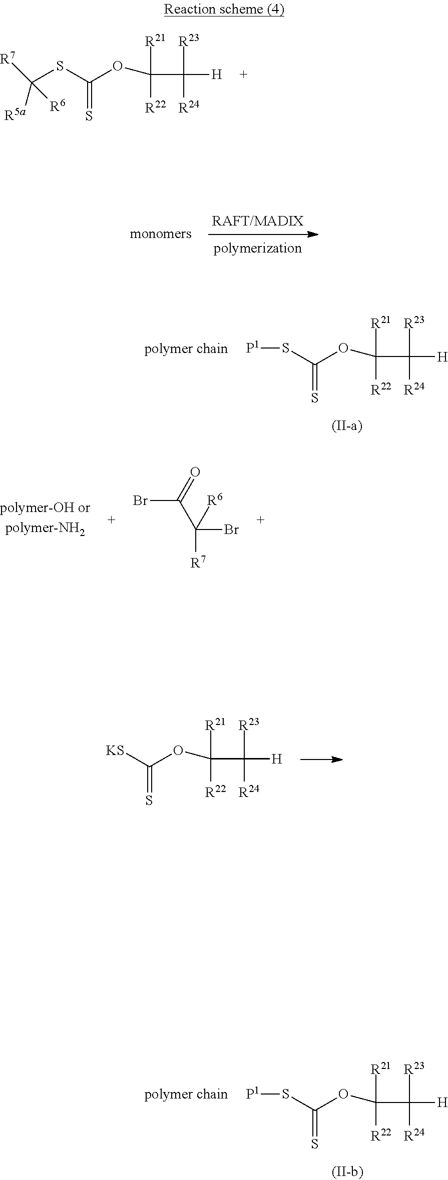

According to one embodiment of the invention, the xanthate of formula (II) is S-(cyanomethyl)-O-(3-methylbutan-2-yl) carbonodithioate (XA1) and S-((1,3-dioxoisoindolin-2-yl)methyl)-O-(3-methylbutan-2-yl) carbonodithioate (XA2).

Stage 1) of preparation of the monoadduct of formula (IV) of the process in accordance with the invention can be carried out without solvent, in water or in an organic solvent. It is preferably carried out in an organic solvent or in water and more preferably still in an organic solvent. The organic solvent which can be used during this stage 1) is then preferably chosen from toluene, tetrahydrofuran (THF), ethyl acetate and 1,4-dioxane. Toluene is particularly preferred among such organic solvents.

Within the meaning of the present invention, radical initiator is understood to mean a chemical entity capable of forming free radicals, that is to say a chemical entity possessing one or more unpaired electrons in its outer shell.

According to the process in accordance with the invention, the radical initiator used during stage 1) is preferably chosen from organic peroxides, azo derivatives and redox systems.

Mention may in particular be made, among organic peroxides, of dilauroyl peroxide (LPO), t-butyl peroxyacetate, t-butyl peroxybenzoate, t-butyl peroxyoctoate, t-butyl peroxydodecanoate, t-butyl peroxyisobutyrate, t-amyl peroxypivalate, t-butyl peroxypivalate, diisopropyl peroxydicarbonate, dicyclohexyl peroxydicarbonate, dicumyl peroxide, dibenzoyl peroxide, potassium peroxydisulfate, sodium peroxydisulfate and ammonium peroxydisulfate. LPO is particularly preferred among these organic peroxides.

Mention may in particular be made, among the azo derivatives, of 2,2′-azobis(isobutyronitrile), 2,2′-azobis(2-cyano-2-butane), dimethyl-2,2′-azobisdimethylisobutyrate, 4,4′-azobis-(4-cyanopentanoic acid), 1,1′-azobis(cyclohexanecarbonitrile), 2-(t-butylazo)-2-cyanopropane, 2,2′-azobis[2-methyl-N(1,1)-bis(hydroxymethyl)-2-hydroxyethyl] propanamide, 2,2′-azobis[2-methyl-N-hydroxyethyl] propanamide, 2,2′-azobis(N,N′-dimethyleneisobutyramidine) dihydrochloride, 2,2′-azobis(2-amidinopropane) dihydrochloride, 2,2′-azobis(N,N′-dimethylene isobutyramine), 2,2′-azobis(2-methyl-N-[1,bis-(hydroxymethyl)-2-hydroxyethyl]propionamide), 2,2′-azobis(2-methyl-N-[1,1-bis(hydroxymethyl)propionamide], 2,2′-azobis[2-methyl-N-(2-hydroxyethyl)propionamide], 2,2′-azobis(isobutyramide) dihydrate, 2,2′-azobis(2,2,4-trimethylpentane) and 2,2′-azobis(2-methylpropane).

The redox systems are, for example, chosen from systems comprising combinations, such as the following combinations:
mixtures of hydrogen peroxide, of a dialkyl peroxide, of hydroperoxide, of a perester, of a percarbonate or similar compounds with an iron salt, a titanium salt, a zinc formaldehyde sulfoxylate salt or a sodium formaldehyde sulfoxylate salt,
mixtures of hydrogen peroxide, of a dialkyl peroxide, of hydroperoxide, of a perester, of a percarbonate or similar compounds with an organic acid, such as ascorbic acid or erythorbic acid,
mixtures of an alkali metal or ammonium persulfate, perborate or perchlorate with an alkali metal bisulfite, such as sodium metabisulfite,
mixtures of an alkali metal or ammonium persulfate, perborate or perchlorate with an organic acid, such as ascorbic acid or erythorbic acid, or
mixtures of an alkali metal persulfate with an arylphosphinic acid, such as benzenephosphonic acid or similar compounds.

Preference is very particularly given, among such redox systems, to the combination of ammonium persulfate and of sodium formaldehyde sulfoxylate.

Moreover, during stage 1), the radical initiator can be added to the reaction medium all at once or in several goes, that is to say portionwise.

According to a preferred embodiment form of the process of the invention, the radical initiator is added to the reaction medium portionwise.

According to one embodiment of the invention, the monoadduct of formula (IV) as obtained on conclusion of stage 1) is chosen from methyl 6-cyano-2-methyl-4-((((3-methylbutan-2-yl)oxy)carbonothioyl)thio)hexanoate (XA1CN) and methyl 6-(1,3-dioxoisoindolin-2-yl)-2-methyl-4-((((3-methylbutan-2-yl)oxy)carbonothioyl)thio)hexanoate (XA2PH).

The monomers of formula (III) are preferably chosen from compounds which are monomers only slightly, or not at all, polymerizable under the temperature and pressure conditions used during stage 1) of the process in accordance with the invention, that is to say which result in a monoadduct of formula (IV) without notable presence of diadduct, triadduct, and the like. Mention may in particular be made, among such monomers of formula (III), of methyl 2-methyl-4-pentenoate (A1).

The monomers of formula (III) are generally commercially available. When they are not commercially available, they can be easily obtained by synthesis routes which are well known to a person skilled in the art.

Stage 1) of the process in accordance with the invention is generally carried out at a temperature varying from 10 to 140° C. approximately, preferably from 40 to 110° C. approximately and more preferably still between 65 and 90° C. approximately.

The duration of said stage 1) generally varies from 3 to 48 hours approximately and more preferably still from 4 to 24 hours approximately.

According to a specific and preferred embodiment of the invention, the monoadduct of formula (IV) obtained on conclusion of stage 1) is purified, for example by silica gel chromatography, before being used in the second thermolysis stage.

The thermolysis stage 2) of the process in accordance with the present invention can be carried out with or without solvent. According to a preferred embodiment of the invention, the thermolysis stage 2) is carried out without solvent. The temperature of the thermolysis stage 2) is generally between 40 and 210° C. approximately, preferably between 100 and 200° C. approximately and more particularly between 160 and 190° C. approximately.

The thermolysis stage 2) is generally carried out at a temperature sufficient to decompose the monoadduct of formula (IV).

In the present invention, the expression “thermolysis” means a thermal decomposition. It is a reaction of thermal decomposition caused by heat. In the present case, the action of the heat results in the decomposition of the monoadduct of formula (IV), making possible the formation of the thiolactone of formula (I).

In other words, stage 2) of the process of the invention does not employ base(s) and/or acid(s) and preferably does not employ reactants other than the monoadduct of formula (IV) resulting from stage 1). In other words, the action of the heat alone makes it possible to result in the thiolactones of formula (I).

Surprisingly, the monoadduct of formula (IV) obtained in stage 1) has a chemical structure suitable, in particular because of the definition of the R¹, R², R³, R⁴, R^{5b}, R⁶, R⁷, R²¹, R²², R²³, R²⁴ and R²⁵ groups, for making possible the formation of a substituted thiolactone of formula (I) by thermolysis. In other words, the cyclization to give thiolactone (I) is favoured.

When the thermolysis stage 2) is carried out in a solvent, then said solvent is preferably chosen from solvents of high boiling point (that is to say, having a boiling point of greater than or equal to the thermolysis temperature), such as, for example, 1,2-dichlorobenzene.

Moreover, the thermolysis stage 2) can be carried out at atmospheric pressure or under vacuum, in particular in the latter case, in order to remove the volatile by-products possibly formed during the reaction.

According to a specific embodiment, the thermolysis stage 2) is carried out in a closed container (e.g. Schlenk tube) and preferably under vacuum.

According to a specific and preferred form of the invention, the thermolysis stage 2) is carried out without solvent and under vacuum.

At the end of the thermolysis stage 2), the thiolactone of formula (I) is preferably purified, for example by silica column chromatography.

Some of the substituted thiolactones of formula (I) which are directly obtained by carrying out the preparation process in accordance with the first subject-matter of the invention are novel per se and as such constitute the second subject-matter of the invention.

Another subject-matter of the present invention is thus substituted thiolactones of following formula (I′):

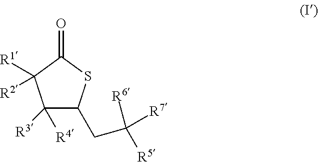

in which:
R^{1′}, R^{2′}, R^{3′} and R^{4′}, which are identical or different, represent a hydrogen atom or a group chosen from alkyl, acyl, aryl, heteroaryl, saturated or unsaturated cycloalkyl and saturated or unsaturated heterocycloalkyl groups, it also being possible for the R^{1′}, R^{2′}, R^{3′} and R^{4′} radicals to together form a saturated or unsaturated cycloalkyl or heterocycloalkyl group or an aryl or heteroaryl group; and
R^{5′}, R^{6′} and R^{7′} are defined according to one of the following two options (i) or (ii):

(i)
R^{5′} is chosen from a cyano group and a phthalimido group; and
R^{6′} and R^{7′}, which are identical or different, are chosen from a hydrogen atom, an alkyl group, an acyl group, an aryl group, a heteroaryl group, an aralkyl group, a saturated or unsaturated cycloalkyl group and a saturated or unsaturated heterocycloalkyl group, it being possible for said alkyl, acyl, aryl, heteroaryl, aralkyl, saturated or unsaturated cycloalkyl and saturated or unsaturated heterocycloalkyl groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N+R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from CI, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or

(ii) R^{5′}, R^{6′} and R^{7′} are such that they together form a polymer chain P¹.

Option (i) is preferred.

In a specific embodiment, R^{1′}, R^{2′}, R^{3′} and R^{4′} represent a hydrogen atom or an alkyl group.

Preferably, R^{1′} (respectively R^{2′}) is an alkyl group, in particular a methyl group, and R^{2′} (respectively R^{1′}) is a hydrogen atom.

Preferably, at least one of the R^{3′} or R^{4′} groups is a hydrogen atom and more preferably the two R^{3′} and R^{4′} groups are hydrogen atoms.

Preferably, at least one of the R^{6′} or R^{7′} groups is a hydrogen atom and more preferably the two R^{6′} and R^{7′} groups are hydrogen atoms.

Mention may in particular be made, among the substituted thiolactones of formula (I′) above, of:
3-(4-methyl-5-oxotetrahydrothiophen-2-yl)propanenitrile (TL1), and
2-(2-(4-methyl-5-oxotetrahydrothiophen-2-yl)ethyl)isoindoline-1,3-dione (TL2).

The substituted thiolactones of formula (I) capable of being obtained by the implementation of the process in accordance with the present invention and in particular the thiolactones of formula (I′) in accordance with the second subject-matter of the present invention can advantageously be used in the synthesis of polymers or in the functionalization of particles, of flat surfaces of metal, glass or ceramic type, or of polymers.

Consequently, the present invention thus also has, as third subject-matter, the use of at least one substituted thiolactone of formula (I′) in the synthesis of polymers or in the functionalization of particles, of flat surfaces of metal, glass or ceramic type, or of polymers.

As regards the preparation of polymers, the thiolactones of formula (I) and in particular of formula (I′) can be used in a polymerization reaction comprising at least one stage of reaction of a thiolactone of formula (I), in particular of formula (I′), with a nucleophilic compound, making it possible to open the ring of the thiolactone and to obtain a thiol which can subsequently be used in an addition or condensation polymerization process with, for example, a monomer of diacrylate type, such as described in the reference by Yu et al. [Polym. Chem., 2015, 6, 1527-1532].

As regards the functionalization of surfaces or of polymers, it is thus possible:
according to a first embodiment, to graft substituted thiolactones of formula (I) (respectively of formula (I′)) to a solid surface or to a polymer in the liquid state, said surface or said polymer comprising chemical functional groups capable of reacting with one of the R¹, R², R³, R⁴, R⁵, R⁶ or R⁷ (respectively R^{1′}, R^{2′}, R^{3′}, R^{4′}, R^{5′}, R^{6′} or R^{7′}) groups of the thiolactones of formula (I) (respectively (I′)), in order to form a covalent bond, or strong interactions of hydrogen bond type. By way of example, it is thus possible to graft a thiolactone comprising a phosphonate group as X substituent of R⁶ or R⁷ to a metal surface. According to this first embodiment, after functionalization, the integrity of the thiolactone ring is preserved.
according to a second embodiment, to graft the substituted thiolactone by opening of the thiolactone ring to a solid surface or to a polymer in the liquid state and reacting with any substance which reacts with thiols, such as alkyl halides or acrylates, for example.

Depending on the nature of the R¹, R², R³, R⁴, R⁵, R⁶ or R⁷ (respectively R^{1′}, R^{2′}, R^{3′}, R^{4′}, R^{5′}, R^{6′} or R^{7′}) groups, it then becomes possible to confer, on a material or on a polymer, the properties corresponding to the type of R¹, R², R³, R⁴, R⁵, R⁶ or R⁷ (respectively R^{1′}, R^{2′}, R^{3′}, R^{4′}, R^{5′}, R^{6′} or R^{7′}) group grafted, for example non-stick properties when the R¹, R², R³, R⁴, R⁵, R⁶ or R⁷ (respectively R^{1′}, R^{2′}, R^{3′}, R^{4′}, R^{5′}, R^{6′} or R^{7′}) groups are perfluorinated groups. It is also possible to introduce an additional functional group during the reacting of the thiol obtained by opening of the thiolactone with a functional compound which reacts with thiols.

The present invention is illustrated by the following implementational examples, to which, however, it is not limited.

### EXAMPLES

### Example 1: Synthesis of 3-(4-methyl-5-oxotetrahydrothiophen-2-yl)propanenitrile (TL1) According to the Process in Accordance with the Invention

In this example, the thiolactone of following formula (TL1) was prepared:

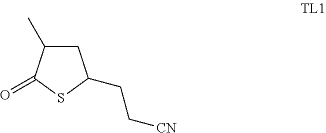

### 1) First Stage: Preparation of S-(cyanomethyl)-O-(3-methylbutan-2-yl) carbonodithioate (Xanthate of Formula (II); (XA1))

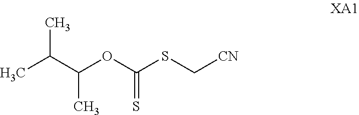

### 1.1) Substage 1: Preparation of Potassium O-1,2-Dimethyl Propyl) Xanthatate (XA0)

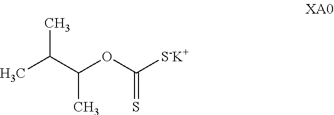

100 g (1.13 mol) of 3-methylbutan-2-ol (Alfa Aesar), 63.65 g of potassium hydroxide (KOH, Sigma-Aldrich) and 90.7 g (1.19 mol) of carbon disulfide (CS₂, Sigma-Aldrich) were suspended in 500 ml of tetrahydrofuran (THF, Sigma-Aldrich) at ambient temperature for 24 hours.

After complete dissolution of the KOH, the yellow emulsion was concentrated under reduced pressure, then triturated with pentane (Sigma-Aldrich) and finally filtered in order to obtain 185 g of the expected product XA0 in the form of a yellow solid (185 g, yield 80%).

¹H NMR (300.13 MHz, D₂O, 298K) δ: 5.31 (p, ³J_{H,H}=6.4 Hz, 1H, (CH₃)₂CHCH(O)CH₃); 2.07-1.84 (m, 1H, (CH₃)₂CHCH(O)CH₃); 1.27 (d, ³J_{H,H}=6.4 Hz, 3H, (CH₃)₂CHCH(O)CH₃); 0.96 (d, ³J_{H,H}=6.9 Hz, 6H, (CH₃)₂CHCH(O)CH₃) ppm.

¹³C{¹H} NMR (75.47 MHz, D₂O, 298K) δ: 232.6 (C═S); 86.3 ((CH₃)₂CHCH(O)CH₃); 32.8 ((CH₃)₂CHCH(O)CH₃); 17.7 ((CH₃)₂CHCH(O)CH₃); 17.6 ((CH₃)₂CHCH(O)CH₃); 15.8 ((CH₃)₂CHCH(O)CH₃) ppm.

### 1.2) Substage 2: Preparation of S-(cyanomethyl)-O-(3-methylbutan-2-yl) Carbonodithioate (XA1)

0.025 mol (5.05 g) of 2-bromoacetonitrile (Sigma-Aldrich) was added to a solution of 3.11 g (0.026 mol) of the compound XA0 obtained above in the preceding stage 1.1) in 25 ml of THF (Sigma-Aldrich), in an ice bath (highly exothermic reaction). Once the addition was complete, the reaction medium was stirred at ambient temperature for 16 hours and then filtered. The filtrate was concentrated under vacuum and the crude reaction product was purified by silica chromatography (eluent petroleum ether/ethyl acetate: 80:20, v:v) in order to recover the xanthate XA1 in the form of a yellow oil (3.58 g, yield 71%).

### 2) Second Stage: Preparation of Methyl 6-cyano-2-methyl-4-((((3-methylbutan-2-yl)oxy)carbonothioyl)thio)hexanoate (Monoadduct of Formula (IV); XA1CN)

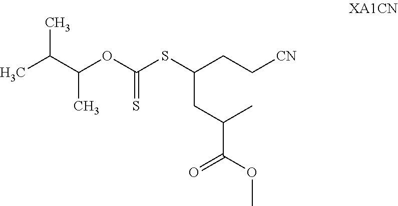 [SMILES: COC(=O)C(C)CC(CCC#N)SC(=S)OC(C)C(C)C]

### 2.1) Substage 1: Preparation of methyl 2-methyl-4-pentenoate (A1)

0.075 mol (10.6 g) of ethyl 2-methyl-4-pentenoate (Sigma-Aldrich), 120 ml of methanol (Sigma-Aldrich) and 0.0038 mol (0.369 g) of 97% sulfuric acid (Sigma-Aldrich) were brought to reflux for 24 hours. The reaction mixture was subsequently cooled, diluted with 100 ml of diethyl ether (Sigma-Aldrich) and extracted with aqueous sodium chloride solution until a neutral pH was reached. The organic phase was dried over magnesium sulfate (Sigma-Aldrich) and evaporated under vacuum. Methyl 2-methyl-4-pentenoate (A1) was obtained in the form of a colourless liquid (7.15 g, yield 75%).

### 2.2) Substage 2: Preparation of methyl 6-cyano-2-methyl-4-((((3-methylbutan-2-yl)oxy)carbonothioyl)thio)hexanoate (XA1CN)

3.45 g (0.17 mmol) of the xanthate XA1 obtained above in the preceding substage 1.2), 2.01 g (0.016 mmol) of methyl 2-methyl-4-pentenoate A1 obtained above in the preceding substage 2.1), 0.90 g (0.0023 mmol) of dilauroyl peroxide (LPO: radical initiator) and 3.5 ml of toluene were mixed in a Schlenk tube. The mixture was subsequently degassed by 3 operations of freezing under vacuum. After heating for 16 hours at a temperature of 90° C., the crude reaction product was purified by silica chromatography (eluent ethyl acetate/hexane (2:8, v:v)) in order to recover the monoadduct XA1CN (3.96 g, yield 79%, yellow oil).

¹H NMR (300.13 MHz, CDCl₃, 298K) δ (ppm): 5.59-5.47 (m, 1H, (CH₃)₂CHCH(O)CH₃), 3.94-3.74 (m, 1H, SCH(CO)CH), 3.67-3.66 (m, 3H, C(O)CH₃), 2.76-2.60 (m, 1H, SCH₂CH(CH₃)), 2.54-2.45 (m, 2H, SCH₂CH₂CN), 2.19-1.93 (m, 4H, SCH(CH₂CH₂CN)CH₂CH(CH₃)), 1.78-1.52 (m, 1H, (CH₃)₂CHCH(O)CH₃), 1.31-1.27 (m, 3H, CH(O)CH₃), 1.20-1.18 (d, 3H, SCH₂CH(CH₃)), 0.95-0.94 (m, 6H, (CH₃)₂CHCH(O)CH₃).

¹³C{¹H} NMR (75.47 MHz, CDCl₃, 298K) δ (ppm): 212.40 (C═S), 176.12 (CO₂CH₃), 119.10 (CN), 86.47 ((CH₃)₂CHCH(O)CH₃), 51.94 (CO₂CH₃), 48.35-47.56 (SCHCH₂CH₂N), 37.72 (SCHCH₂CH₂CN), 37.06 (SCHCH₂CH(CH₃)), 32.70 ((CH₃)₂CHCH(O)CH₃), 31.62-30.90 (SCH(CH₂CH(CH₃)), 18.28-15.75 ((CH₃)₂CHCH(O)CH₃) and SCHCH₂CH(CH₃)), 14.78 (SCH₂CH₂CN).

IR: 2971, 2876, 2247, 1734, 1460, 1237, 1044 cm⁻¹.

Molar mass: CI (CH₄), MH⁺:

Found: 332.1369 g/mol,

Calculated: 332.1354 g/mol.

### 3) Third Stage: Preparation of 3-(4-methyl-5-oxotetrahydrothiophen-2-yl)propanenitrile (Thiolactone of Formula (I); TL1)

2.05 g (0.0062 mol) of XA1CN obtained above in the preceding substage 2.2) were placed in a Schlenk tube closed under vacuum and were brought to a temperature of 190° C. for 24 hours. The reaction mixture was subsequently cooled to ambient temperature and the volatile compounds formed were removed under vacuum. The thiolactone TL1 thus obtained in the form of a colourless oil was subsequently purified on a silica chromatography column (eluent hexane/ethyl acetate: 6:4 (v:v)) (0.42 g, yield 40%).

¹H NMR (300.13 MHz, CDCl₃, 298K) δ (ppm): 3.87-3.73 (m, 1H, CH(CH₂)₂CN), 3.69-1.39 (m, 7H, C(O)CH(CH₃)CH₂CH(CH₂)₂), 1.10-1.07 (m, 3H, CHCH₃).

¹³C{¹H} NMR (75.47 MHz, CDCl₃, 298K) δ (ppm): 209.06-207.98 (C═O), 118.76 (CN), 48.69 (CHCH₃), 45.46-44.88 (CH(CH₂)₂CN), 40.30-38.74 (CH(CH₃)CH₂CH), 32.09-31.67 (CH(CH₂CH₂CN), 16.10-15.06 (CH(CH₂CH₂CN), 15.12-14.34 (CHCH₃).

IR: 2970, 2875, 2247, 1701, 1453, 756 cm⁻¹.

Molar mass: CI (CH₄), MH⁺:

Found: 170.0643 g/mol,

Calculated: 170.0640 g/mol.

### Example 2: Synthesis of 2-(2-(4-methyl-5-oxotetrahydrothiophen-2-yl)ethyl)isoindoline-1,3-dione (TL2) According to the Process in Accordance with the Invention

In this example, the thiolactone of following formula (TL2) was prepared:

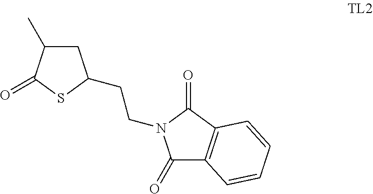 [SMILES: CC1CC(CCN2C(=O)c3ccccc3C2=O)SC1=O]

### 1) First Stage: Preparation of S-((1,3-dioxoisoindolin-2-yl)methyl)O-(3-methylbutan-2-yl) Carbonodithioate (Xanthate of Formula (II); (XA2))

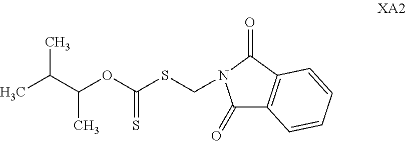 [SMILES: CC(C)C(C)OC(=S)SCN1C(=O)c2ccccc2C1=O]

0.021 mol (4.99 g) of 2-(bromomethyl)-1H-isoindole-1,3(2H)-dione (Sigma-Aldrich) was added to a solution of 4.04 g (0.020 mol) of the compound XA0 obtained in substage 1.1) of Example 1 in 35 ml of acetone (Sigma-Aldrich), in an ice bath (highly exothermic reaction). Once the addition was complete, the reaction medium was stirred at ambient temperature for 3 hours and then filtered. The filtrate was concentrated under vacuum in order to obtain the expected product XA2 in the form of a yellow oil (5.84 g, yield 91%) which will be used in the following stage without purification.

### 2) Second Stage: Preparation of methyl 6-(1,3-dioxoisoindolin-2-yl)-2-methyl-4-((((3-methylbutan-2-yl)oxy)carbonothioyl)thio)hexanoate (Monoadduct of Formula (IV); XA2PH)

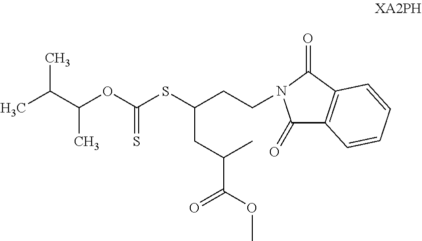 [SMILES: COC(=O)C(C)CC(CCN1C(=O)c2ccccc2C1=O)SC(=S)OC(C)C(C)C]

3.82 g (0.012 mol) of the xanthate XA2 obtained above in the preceding stage 1), 1.38 g (0.012 mol) of methyl 2-methyl-4-pentenoate A1 obtained above in stage 2.1) of Example 1 and 0.64 g (0.0016 mol) of dilauroyl peroxide (LPO: radical initiator) were mixed in a Schlenk tube. The mixture was subsequently degassed by 3 operations of freezing under vacuum. After heating for 16 hours at a temperature of 90° C., the crude reaction product was purified by silica chromatography (eluent ethyl acetate/hexane (2:8, v:v)) in order to recover the unreacted xanthate XA2, on the one hand, and the monoadduct XA2PH, on the other hand (1.9 g, yield 91%, yellow oil).

¹H NMR (300.13 MHz, CDCl₃, 298K) δ (ppm): 7.86-7.67 (m, 4H, Hₐᵣ), 5.56-5.40 (m, 1H, (CH₃)₂CHCH(O)CH₃), 5.22-4.07 (m, 1H, SCH(CO)CH), 3.86-3.70 (m, 3H, SCHCH₂CH₂N), 3.67-3.62 (m, 3H, C(O)CH₃), 2.80-2.60 (m, 1H, SCHCH₂CH(CH₃)), 2.26-1.26 (m, 5H, SCH(CH₂CH₂N—)CH₂CH(CH₃)), 1.29-1.15 (m, 6H, CH(O)CH₃ and SCH₂CH(CH₃)), 0.92-0.86 (m, 6H, (CH₃)₂CHCH(O)CH₃).

¹³C{¹H} NMR (75.47 MHz, CDCl₃, 298K) δ (ppm): 213.17 (C═S), 176.41 (CO₂CH₃), 168.22 (C(O)NC(O), 133.90-123.24 (Cₐᵣ), 85.75 ((CH₃)₂CHCH(O)CH₃), 51.77 (CO₂CH₃), 46.87-46.17 (SCHCH₂CH₂N), 37.18 (SCHCH₂CH(CH₃)), 37.91-33.56 (SCH(CH₂)₂N)CH₂CH(CH₃)), 32.69 ((CH₃)₂CHCH(O)CH₃), 18.26-15.77 ((CH₃)₂CHCH(O)CH₃) and SCHCH₂CH(CH₃)).

IR: 2970, 1773, 1714, 1398, 1234, 1046, 721 cm⁻¹.

Molar mass: CI (CH₄), MH⁺:

Found: 452.1572 g/mol,

Calculated: 452.1565 g/mol.

### 3) Third Stage: Preparation of 2-(2-(4-methyl-5-oxotetrahydrothiophen-2-yl)ethyl)isoindoline-1,3-dione (Thiolactone of Formula (I); TL2)

1.67 g (0.0037 mol) of XA2PH obtained above in the preceding stage 2) were placed in a Schlenk tube closed under vacuum and were brought to a temperature of 190° C. for 24 hours. The reaction mixture was subsequently dissolved in 3 ml of ethyl acetate and then precipitated from 200 ml of hexane. The white solid obtained was filtered on a Bichner funnel. The thiolactone TL2 was thus obtained in the form of a white solid (0.310 g, yield 30%).

¹H NMR (300.13 MHz, CDCl₃, 298K) δ (ppm): 7.77-7.64 (m, 4H, Hₐᵣ), 3.80-3.61 (m, 3H, CH(CH₂CH₂N), 2.68-1.40 (m, 5H, C(O)CH(CH₃)CH₂CH(CH₂CH₂N), 1.11-1.06 (m, 3H, CHCH₃).

¹³C{¹H} NMR (75.47 MHz, CDCl₃, 298K) δ (ppm): 210.0-209.0 (C(O)S), 168.19 (N(C(O)CH)₂), 134.17-123.33 (Cₐᵣ), 48.33 (CH(CH₃)), 45.00-44.56 (CH₂CH(CH₂CH₂N)), 40.87-39.15 (CH₂CH(CH₂CH₂N)), 36.47-35.40 (CH₂CH(CH₂CH₂N)), 15.20-14.38 (CH(CH₃)).

IR: 2936, 1705, 1699, 1399, 1370, 724 cm⁻¹.

Molar mass: CI (CH₄), MH⁺:

Found: 289.0782 g/mol, Calculated: 289.0773 g/mol.

1. Process for the preparation of substituted thiolactones of following formula (I): 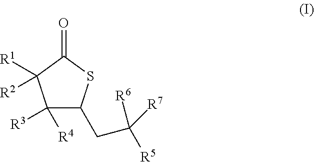
in which:
R¹, R², R³ and R⁴, which are identical or different, represent a hydrogen atom or a group chosen from alkyl, acyl, aryl, heteroaryl, saturated or unsaturated cycloalkyl and saturated or unsaturated heterocycloalkyl groups, it also being possible for the R¹, R², R³ and R⁴ radicals to together form a saturated or unsaturated cycloalkyl or heterocycloalkyl group or an aryl or heteroaryl group; and
R⁵, R⁶ and R⁷, which are identical or different, are:
(a) chosen from a hydrogen atom, a cyano (CN) group, an alkyl group, an acyl group, an aryl group, a heteroaryl group, an aralkyl group, a saturated or unsaturated cycloalkyl group, a saturated or unsaturated heterocycloalkyl group and a phthalimido group, it being possible for said alkyl, acyl, aryl, heteroaryl, aralkyl, saturated or unsaturated cycloalkyl, saturated or unsaturated heterocycloalkyl and phthalimido groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N⁺R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from Cl, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or
(b) such that they together form a polymer chain P¹, or
(c) such that R⁵ is different from the other two groups R⁶ and R⁷, R⁶ and R⁷ have the same definitions as in the alternative (a) and R⁵ is a thiolactone radical of following formula: 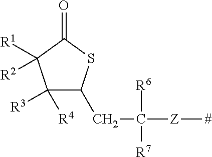
in which R¹, R², R³ and R⁴ have the same meanings as in the formula (I) above, R⁶ and R⁷ have the same definitions as in the alternative (a), Z represents a divalent group chosen from a carbonyl group, a carbonate group, an alkylene group and an arylene group, and the sign # represents the point of attachment of the thiolactone radical to the —CR⁶R⁷CH₂-thiolactone radical of the compound of formula (I);
wherein said comprises at least the following stages:
1) a stage during which a xanthate of following formula (II): 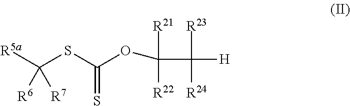
in which:
R²¹, R²², R²³ and R²⁴, which are identical or different, represent a hydrogen atom or a group chosen from alkyl, acyl, aryl, heteroaryl, alkenyl, alkynyl, saturated or unsaturated cycloalkyl and saturated or unsaturated heterocycloalkyl groups, it also being possible for the R²¹, R²², R²³ and R²⁴ radicals to together form a saturated or unsaturated cycloalkyl or heterocycloalkyl group or an aryl or heteroaryl group;
and
R^{5a}, R⁶ and R⁷, which are identical or different, are:
(a′) chosen from a hydrogen atom, a cyano (CN) group, an alkyl group, an acyl group, an aryl group, a heteroaryl group, an aralkyl group, a saturated or unsaturated cycloalkyl group, a saturated or unsaturated heterocycloalkyl group and a phthalimido group, it being possible for said alkyl, acyl, aryl, heteroaryl, aralkyl, saturated or unsaturated cycloalkyl, saturated or unsaturated heterocycloalkyl and phthalimido groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N+R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from Cl, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or
(b′) such that they together form a polymer chain P¹; or
(c′) such that R^{5a} is different from the other two groups R⁶ and R⁷, R⁶ and R⁷ have the same definitions as in the alternative (a′) and R^{5a} is a xanthate radical of formula: 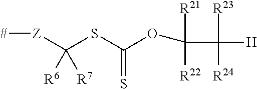
in which R²¹, R²², R²³ and R²⁴ have the same meanings as in the formula (II) above, R⁶ and R⁷ have the same definitions as in the alternative (a′), Z represents a divalent group chosen from a carbonyl group, a carbonate group, an alkylene group and an arylene group, and the sign # represents the point of attachment of the xanthate radical to the —CR⁶R⁷-xanthate radical of the compound of formula (II);
is reacted, in the presence of a radical initiator, with a monomer comprising at least one ethylenic unsaturation of following formula (III): 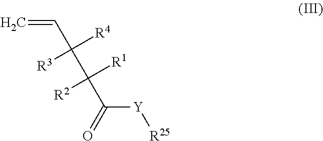
in which:
R²⁵ represents a group chosen from alkyl, acyl, aryl, heteroaryl, aralkyl, saturated or unsaturated cycloalkyl and saturated or unsaturated heterocycloalkyl groups;
Y is an oxygen atom or an NR²⁶ radical in which R²⁶ represents a hydrogen atom or an alkyl group, and preferably an oxygen atom; and
R¹, R², R³ and R⁴ have the same meanings as in the formula (I) above;
to form a monoadduct of following formula (IV): 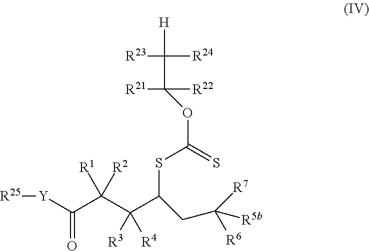
in which:
R¹, R², R³ and R⁴ have the same meanings as in the formula (I) above;
R²¹, R²², R²³ and R²⁴ have the same meanings as in the formula (II) above; and
R²⁵ has the same meaning as in the formula (III) above; and
R^{5b}, R⁶ and R⁷, which are identical or different, are:
(a″) chosen from a hydrogen atom, a cyano (CN) group, an alkyl group, an acyl group, an aryl group, a heteroaryl group, an aralkyl group, a saturated or unsaturated cycloalkyl group, a saturated or unsaturated heterocycloalkyl group and a phthalimido group, it being possible for said alkyl, acyl, aryl, heteroaryl, aralkyl, saturated or unsaturated cycloalkyl, saturated or unsaturated heterocycloalkyl and phthalimido groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N+R¹⁵R^{15′}R^{15″}A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from Cl, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or
(b″) such that they together form a polymer chain P¹; or
(c″) such that R^{5b} is different from the other two groups R⁶ and R⁷, R⁶ and R⁷ have the same definitions as in the alternative (a″), and R^{5b} is a monoadduct radical of following formula: 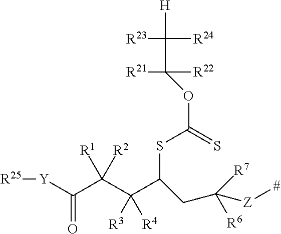
in which R¹, R², R³, R⁴, R²¹, R²², R²³, R²⁴, Y and R²⁵, have the same meanings as in the formula (IV) above, R⁶ and R⁷ have the same definitions as in the alternative (a″), Z represents a divalent group chosen from a carbonyl group, a carbonate group, an alkylene group and an arylene group, and the sign # represents the point of attachment of the monoadduct radical to the —CR⁶R⁷-monoadduct radical of the compound of formula (IV); then
2) a stage of thermolysis of the monoadduct of formula (IV) obtained above in the preceding stage in order to form a corresponding substituted thiolactone of formula (I).

2. Process according to claim 1, wherein R²¹, R²², R²³ and R²⁴ represent a hydrogen atom or an alkyl group.

3. Process according to claim 1, wherein R⁵ is a cyano group or a phthalimido group.

4. Process according to claim 1, wherein R²⁵ is an alkyl group.

5. Process according to claim 1, wherein R¹, R², R³ and R⁴ represent a hydrogen atom or an alkyl group.

6. Process according to claim 1, wherein said process results in the formation of a thiolactone of formula (I) chosen from:
3-(4-methyl-5-oxotetrahydrothiophen-2-yl)propanenitrile (TL1), and
2-(2-(4-methyl-5-oxotetrahydrothiophen-2-yl)ethyl)isoindoline-1,3-dione (TL2).

7. Process according to claim 1, wherein stage 1) of preparation of the monoadduct of formula (IV) is carried out without solvent, in water or in an organic solvent.

8. Process according to claim 1, wherein wherein the radical initiator used during stage 1) is chosen from organic peroxides, azo derivatives and redox systems.

9. Process according to claim 1, wherein stage 1) is carried out at a temperature varying from 10 to 140° C.

10. Process according to claim 1, wherein the thermolysis stage 2) is carried out without solvent.

11. Process according to claim 1, wherein the temperature of the thermolysis stage 2) is between 40 and 210° C.

12. Process according to claim 1, wherein the thermolysis stage 2) is carried out in a closed container and under vacuum.

13. Substituted thiolactones obtained according to a process as defined in claim 1 and corresponding to the following formula (I′): 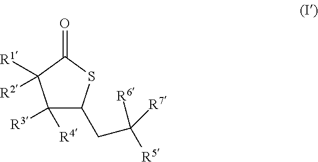
in which:
R^{1′}, R^{2′}, R^{3′} and R^{4′}, which are identical or different, represent a hydrogen atom or a group chosen from alkyl, acyl, aryl, heteroaryl, saturated or unsaturated cycloalkyl and saturated or unsaturated heterocycloalkyl groups, it also being possible for the R^{1′}, R^{2′}, R^{3′} and R^{4′} radicals to together form a saturated or unsaturated cycloalkyl or heterocycloalkyl group or an aryl or heteroaryl group; and
R^{5′}, R^{6′} and R^{7′} are defined according to one of the following two options (i) or (ii):
(i)
R^{5′} is chosen from a cyano group and a phthalimido group; and
R^{6′} and R^{7′}, which are identical or different, are chosen from a hydrogen atom, an alkyl group, an acyl group, an aryl group, a heteroaryl group, an aralkyl group, a saturated or unsaturated cycloalkyl group and a saturated or unsaturated heterocycloalkyl group, it being possible for said alkyl, acyl, aryl, heteroaryl, aralkyl, saturated or unsaturated cycloalkyl and saturated or unsaturated heterocycloalkyl groups to be substituted by an X group chosen from the following groups: P(O)(OR⁸)(OR^{8′}), in which the R⁸ and R^{8′} radicals, which are identical or different, represent a hydrogen atom or an alkyl radical; CₙF₂ₙ₊₁, in which n is an integer ranging from 1 to 20; SiR⁹ₚ(OR¹⁰)₃₋ₚ, in which the R⁹ and R¹⁰ radicals, which are identical or different, represent a hydrogen atom or an alkyl radical and p is an integer equal to 0, 1 or 2; BF₃M, in which M=K or Na; B(OR¹¹)₂, in which the two R¹¹ radicals, which are identical or different, represent a hydrogen atom, an alkyl radical or form a carbon-based ring with the two oxygen atoms to which they are bonded; OR¹², in which R¹² represents a hydrogen atom or an alkyl, aryl or aralkyl radical; O(C═O)R¹³, in which R¹³ represents a hydrogen atom or an alkyl, aryl or aralkyl radical; O(C═O)OR¹⁴, in which R¹⁴ represents a hydrogen atom or an alkyl, aryl or aralkyl radical; N+R¹⁵R^{15′}R^{15″} A⁻, in which the R¹⁵, R^{15′} and R^{15″} radicals, which are identical or different, represent a hydrogen atom or an alkyl, aryl or aralkyl radical and A represents a chlorine or bromine atom; NR¹⁶(C═O)R^{16′}, in which the R¹⁶ and R^{16′} radicals, which are identical or different, represent a hydrogen atom or an alkyl or aryl radical or are connected together and form a ring, such as a pyrrolidone or caprolactam ring; NR¹⁷(C═O)OR^{17′}, in which R¹⁷ and R^{17′}, which are identical or different, represent a hydrogen atom or an alkyl, aryl or aralkyl radical; CN; a halogen atom chosen from Cl, F and Br; NCS; OCH₂-epoxy; COOR¹⁸, in which R¹⁸ represents a hydrogen atom or an alkyl, aryl or aralkyl radical; CONR¹⁹R^{19′}, in which R¹⁹ and R^{19′}, which are identical or different, represent a hydrogen atom or an alkyl or aryl radical; SO₂R²⁰, in which R²⁰ represents an alkyl or aryl radical; azide (N₃) and alkynyl; or
(ii) R^{5′}, R^{6′} and R^{7′} are such that they together form a polymer chain P¹.

14. Thiolactones according to claim 13, wherein they are chosen from:
3-(4-methyl-5-oxotetrahydrothiophen-2-yl)propanenitrile (TL1), and
2-(2-(4-methyl-5-oxotetrahydrothiophen-2-yl)ethyl)isoindoline-1,3-dione (TL2).

15. Polymers or functionalized particles, of flat surfaces or of polymers, comprising at least one substituted thiolactone of formula (I′) as defined in claim 13.
